(19) United States
(12) Patent Application Publication
(10) Pub. No.: US 2009/0182054 A1
(43) Pub. Date: Jul. 16, 2009

(54) Topical compositions containing solubilized dicarboxylic acids

(76) Inventor: Jerry Zhang, Grayslake, IL (US)

Correspondence Address: JERRY ZHANG, 1061 CHADWICK DR., GRAYSLAKE, IL, 60030, US

(21) Appl. No.: 12/319,533

(22) Filed: Jan. 8, 2009

Related U.S. Application Data
(60) Provisional application No. 61/004,113, filed on Jan. 10, 2008.

(51) Int. Cl. A61K 31/194; A61P 17/10

(52) U.S. Cl. 514/574

(57) ABSTRACT

Topical compositions containing at least one solubilized dicarboxylic acid having 6 to 12 carbon atoms are prepared in pharmaceutically acceptable carriers in the presence of at least one 1,2-alkanediol having 5 to 7 carbon atoms. In particular, a topical composition containing solubilized azelaic acid is formulated in an aqueous carrier. In-vitro skin penetration and bioavailability study demonstrates higher bioavailability of the solubilized azelaic acid in the cutaneous organs. The topical composition is advantageous in treatment of skin disorders associated with skin inflammation such as rosacea, seborrheic dermatitis, perioral dermatitis, and facial dermatitis.

### CROSS-REFERENCE TO RELATED APPLICATIONS

This application claims the benefit of provisional patent application Ser. No. 61/004,113, filed on Jan. 10, 2008, by the present inventor. It is hereby incorporated by reference in its entirety.

### FEDERALLY SPONSORED RESEARCH

Not Applicable

### SEQUENCE LISTING OR PROGRAM

Not Applicable

### FIELD OF INVENTION

This invention relates to topical compositions containing at least one solubilized dicarboxylic acid having 6 to 12 carbon atoms. In particular, the invention relates to topical compositions for treating skin disorders associated with skin inflammation such as acne, rosacea, seborrheic dermatitis, perioral dermatitis, and facial dermatitis.

### BACKGROUND OF THE INVENTION

Skin disorders such as acne, rosacea, seborrheic dermatitis, and other inflammatory skin conditions are often characterized by facial erythema. For example, rosacea is a chronic disease affecting central face and across the cheeks, nose, or forehead causing skin redness, prominent vascularization, papules, pustules and swelling, as well as predisposition to flushing and blushing.

Several cosmetic and medical treatments have been used in an attempt to treat skin disorders associated with skin inflammation such as acne, rosacea, seborrheic dermatitis, perioral dermatitis, and facial dermatitis.

Azelaic acid, a naturally occurring dicarboxylic acid having 9 carbon atoms, has been used for treatment of rosacea and acne. For example, a commercially available azelaic acid product, Finacea™ (Intendis, Pine Brook, N.J.), is a prescription gel product containing 15% of azelaic acid for treatment of rosacea. Topical application of azelaic acid has been shown to significantly reduce inflammatory papule lesions and erythema. However, azelaic acid in this prescription product is not solubilized but rather in the form of un-solubilized, suspended solids in a gel matrix. In general, active ingredient in the solubilized form is more bioavailable than in the un-solubilized form.

Generally, dicarboxylic acids having from 6 to 12 carbon atoms have limited solubility in aqueous solutions. For example, azelaic acid has a solubility of 0.24 gram per 100 gram of water at 25.degee. C. At this concentration, its therapeutic efficacy is minimal.

U.S. Pat. No. 6,734,210, discloses topical compositions containing solubilized azelaic acid in the presence of polycations such as chitosan, a linear polysaccharide composed of linked D-glucosamine and N-acetyl-D-glucosamine. According to this disclosure, azelaic acid forms stable, polymeric salts with chitosan. However, there is no discussion if azelaic acid in this composition is more bioavailable than the un-solubilized form.

There is a need for a safe and effective topical treatment of skin disorders associated with skin inflammation such as acne, rosacea, seborrheic dermatitis, perioral dermatitis, and facial dermatitis without disadvantages of the prior art.

There is also a need for an enhanced therapy for the skin disorders associated with skin inflammation by employing a topical composition containing solubilized dicarboxylic acid.

### SUMMARY OF THE INVENTION

Accordingly, it is an object of the invention to provide a method for treating skin disorders associated with skin inflammation by topically administrating to a patient in need of such treatment a safe and effective amount of a composition containing at least one solubilized dicarboxylic acid having 6 to 12 carbon atoms in a pharmaceutically acceptable carrier.

Another object of the invention is to formulate topical compositions containing at least one solubilized dicarboxylic acid having 6 to 12 carbon atoms in the presence of at least one 1,2-alkanediol having 5 to 7 carbon atoms.

A further object of the invention is to formulate topical compositions containing solubilized dicarboxylic acids in pharmaceutically acceptable carriers for treating dermatological disorders such as rosacea, seborrheic dermatitis, perioral dermatitis, and facial dermatitis.

Still other objects and advantages of the invention will, in part, be obvious and will, in part, be apparent from the following detailed description of the preferred embodiments.

### BRIEF DESCRIPTIONS OF THE DRAWINGS

FIG. 1 shows chemical structure of dicarboxylic acids.

FIG. 2 shows chemical structure of 1,2,-pentanediol, 1,2-hexanediol, and 1,2-heptanediol.

FIG. 3 is a graph of the percent of applied dose of azelaic acid in FINACEA and a formulation according to the present invention retained on different skin locations and penetrated across the skin into the receptor medium after 8 hours. The error bars represent the standard deviation.

FIG. 4 is a graph of the percent of applied dose of azelaic acid in FINACEA and a formulation according to the present invention retained on different skin locations and penetrated across the skin into the receptor medium after 12 hours. The error bars represent the standard deviation.

FIG. 5 is a graph of the percent of applied dose of azelaic acid in FINACEA and a formulation according to the present invention retained on different skin locations and penetrated across the skin into the receptor medium after 16 hours. The error bars represent the standard deviation.

FIG. 6 is a graph of the percent of applied dose of azelaic acid in FINACEA and a formulation according to the present invention retained on different skin locations and penetrated across the skin into the receptor medium after 20 hours. The error bars represent the standard deviation.

FIG. 7 is a graph of the percent of applied dose of azelaic acid in FINACEA and a formulation according to the present invention retained on different skin locations and penetrated across the skin into the receptor medium after 24 hours. The error bars represent the standard deviation.

### DETAILED DESCRIPTION OF THE PREFERRED EMBODIMENTS

It has been unexpectedly discovered that topical compositions containing solubilized dicarboxylic acid are able to be obtained in the presence of at least one 1,2-alkanediol in pharmaceutically acceptable carriers.

In the described embodiments, disclosed are topical compositions containing at least one solubilized dicarboxylic acid having 6 to 12 carbon atoms and at least one 1,2-alkanediol where the alkane is a pentane, hexane or heptane. Also disclosed are methods for treating skin disorders associated with skin inflammation such as rosacea, seborrheic dermatitis, perioral dermatitis, and facial dermatitis.

As used herein, the term ‘about’ will be understood by persons of ordinary skill in the art and will vary to some extent on the context in which is used. If there are uses of the term which are not clear to persons of ordinary skill in the art given the context in which is used, ‘about’ will mean up to plus or minus 10% of the particular term.

The term ‘dissolved’, ‘dissolving’, ‘solubilized’ or ‘solubilizing’, when used in accordance with the present invention, means that an ingredient is solubilized in a topical composition, and that the ingredient will not exist to any appreciable degree in the particulate, crystalline or droplet form in the composition.

The term ‘pharmaceutically acceptable’, as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term ‘safe and effective’, as used herein, means a concentration of an active ingredient or an amount of a composition, that is sufficient enough to significantly and positively modify the condition to be treated but low enough to avoid serious side effects, within the scope of sound medical advice.

The term ‘dicarboxylic acid’, when used in accordance with the present invention, means that dicarboxylic acid is either prepared from synthetic method or isolated from natural source, either in admixture or in pure or substantially pure form. All physical forms of the dicarboxylic acid, including crystalline, semi-crystalline, and amorphous, are contemplated and within the scope of the present invention, either in admixture or in pure or substantially pure form.

All percentages referred to in this specification are percentages by weight of the total composition unless otherwise indicated.

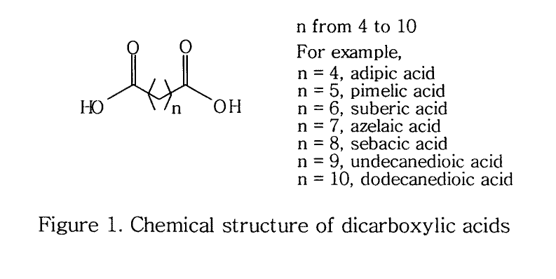

Chemical structure of a dicarboxylic acid in accordance with the present invention is shown in FIG. 1. The alkyl chain in between the two terminal carboxylic groups is a saturated straight alkyl group having 4 to 10 carbon atoms. Examples of the suitable dicarboxylic acid are adipic acid, pimelic acid, suberic acid, azelaic acid, sebacic acid, undecanedioic acid, dodecanedioic acid, and any suitable combinations thereof. These dicarboxylic acids are commercially available from various vendors, for example, Aldrich Chemical Company (Milwaukee, Wis.). Azelaic acid is preferred dicarboxylic acid.

Concentration of the dicarboxylic acid in the composition of the present invention might be in the range of about 1% to about 35%, preferably about 2% to about 30%, more preferably about 3% to about 25%, still more preferably from about 5% to about 20%.

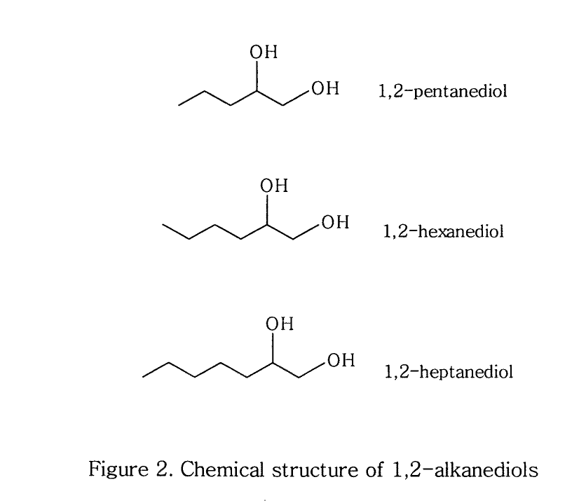

Chemical structure of 1,2-alkanediols having 5 to 7 carbon atoms in accordance with the present invention is shown in FIG. 2. The alkyl chain in the 1,2-alkanediols is a saturated straight alkyl group having 5 to 7 carbon atoms. The 1,2-alkanediols in accordance with the present invention are 1,2-pentanediol, 1,2-hexanediol, 1,2-heptanediol, and any suitable combinations thereof. These 1,2-alkanediols are commercially available, for example, from Aldrich Chemical Company (Milwaukee, Wis.). They may also be chemically synthesized, for example, by catalytic hydroxylation of the corresponding terminal alkenes using a combination of hydrogen peroxide and osmium tetroxide. 1,2-Hexanediol is preferred.

Concentration of the 1,2-alkanediol in the composition of the present invention might be in the range of about 1% to about 50%, preferably about 2% to about 40%, more preferably about 5% to about 35%, still more preferably about 7% to about 30%.

The pH of the composition of the present invention may preferably be kept in the acidic range, meaning the pH value is preferably less than about 6.5. Any pharmaceutically acceptable inorganic or organic base may be used to adjust pH of the composition. Examples of the suitable bases are sodium hydroxide, potassium hydroxide, ammonium hydroxide, triethanolamine, tromethamine, 2-amino-2-methyl-1-propanol, 2-amino-2-methyl-1,3-propanediol, and L-arginine. Also suitable are the compounds that can form complexes with dicarboxylic acids. The complexing compounds in accordance with the present invention are compounds having at least one amide bond. Examples of the suitable complexing compounds are niacinamide, isonicotinamide, urea, methyl urea, 1-butyl urea, and N-2-hydroxyethyl urea. The complexing compound might be added in a concentration range of about 1% to about 20%.

The suitable combinations of the bases and complexing compounds may also be used to adjust pH value of the composition. In one embodiment, a combination of tromethamine and niacinamide may be used. In another embodiment, a combination of triethanolamine and niacinamide may be used. In yet another embodiment, a combination of tromethamine and isonicotinamide may be used. In still another embodiment, a combination of triethanolamine and isonicotinamide may be used.

The pH of the aqueous composition may be kept in the range of about 3.0 to about 6.5, preferably about 3.5 to about 6.0, more preferably about 4.0 to about 5.5.

Dicarboxylic acid and 1,2-alkanediol in the composition of the present invention are solubilized in a pharmaceutically acceptable carrier.

The composition containing a combination of dicarboxylic acid and 1,2-alkanediol might be formulated in any pharmaceutically acceptable carrier suitable for topical application to dermal tissues. The carrier might be aqueous-based or organic solvent-based. The aqueous-based carrier is preferred.

The composition, in accordance with the present invention, may be in the form of a solution, gel, spray, or foam. In one embodiment, the composition is a gel. Therefore, the aqueous composition preferably contains a gelling agent. Any gelling agent that is dispersible in the carrier, physically and chemically compatible with the ingredients in the composition, and forms a gel of substantially uniform consistency is suitable for use in the present invention.

Examples of the suitable gelling agents are inorganic based gelling agents, polycarbohydrate based gelling agents, and polyacrylic acid based gelling agents. Examples of the suitable inorganic gelling agents are silica gel and clay. Examples of the suitable polycarbohydrate based gelling agents are hydroxyethyl cellulose, hydroxypropyl cellulose, and xanthan gum. Examples of the suitable polyacrylic acid based gelling agents are CARBOPOL Brand 934, 940, 941, Ultrez 10, and Ultrez 20 (available from Noveon Corp., Cleveland, Ohio). Combinations of the above types of gelling agents are also suitable as the gelling agents.

The composition, in accordance with the present invention, may optionally contain nonionic surfactants. Nonionic surfactants are generally compounds formed by the condensation of alkylene oxide groups such as, for example, ethylene oxide and propylene oxide, with a lipophilic compound. Examples of classes of nonionic surfactants are:

(a). Polysorbates, or sorbitol or sucrose esters of fatty acids. Examples of the suitable polysorbates are polysorbate-20, polysorbate-40, polysorbate-60, polysorbate-65, polysorbate-80, and polysorbate-85. Examples of the suitable sorbitol esters of fatty acids are sorbitan monooleate, sorbitan monopalmitate, sorbitan monolaurate, sorbitan monostearate, sorbitan monoisostearate, sorbitan sesquioleate, sorbitan trioleate, sorbitan tristearate, sorbitan monooleate ethoxylate EO 20 mole, sorbitan monolaurate ethoxylate EO 20 mole, sorbitan monopalmitate ethoxylate EO 20 mole, sorbitan monostearate ethoxylate EO 20 mole, and so on.
(b). Alkyl polysaccharides having a lipophilic group of 6 to 30 carbon atoms and polysaccharide group such as glucose, galactose, and so on. Examples of the suitable alkyl polysaccharides are octyl, nonydecyl, undecyl, dodecyl, hexadecyl, octadecyl gluocosides, galactosides, and so on.
(c). Polyol or polyethylene glycol (PEG) esters of fatty acids and PEG ethers of fatty alcohols. Examples of the suitable fatty esters or ethers are glyceryl stearate, glyceryl distearate, PEG-40 stearate, PEG-50 stearate, PEG-100 stearate, oleth-5, oleth-10, oleth-20, laureth-23, ceteareth-20, ceteareth-21, steareth-10, steareth-21, and so on.

The composition may contain keratolytic agents. Example of the suitable keratolytic agents are sulfur and allantoin. Concentration of allantoin in the composition of the present invention might be in the range of about 0.2% to about 5%. Concentration of sulfur in the composition of the present invention might be in the range of about 1% to about 10%.

The composition may contain zinc-containing compounds. Examples of the zinc-containing compounds include zinc sulfate, zinc pyrithione, zinc lactate, and zinc gluconate.

The composition may contain .alpha.- and .beta.-hydroxy acids. Examples of the .alpha.-hydroxyl acids, are glycolic acid, lactic acid, and mandelic acid. Example of the beta.-hydroxy acid is salicylic acid.

The composition may contain anti-inflammatory agents. Examples of the suitable anti-inflammatory agents are salicylic acid and pharmaceutically acceptable salts thereof, ketoprofen, ibuprofen, cat's claw extract, green tea extract, curcumin extract, and dandelion extract.

The composition, in accordance with the present invention, may contain conventional amounts of customary auxiliaries and additives provided that such auxiliaries and additives are physically and chemically compatible with the ingredients in the composition, solubilized in the composition, and do not reduce the therapeutic efficacy of the composition.

Furthermore, the auxiliaries and additives may not impair favorable cosmetic properties of the composition of the present invention.

Examples of customary auxiliaries and additives include, but not limited to: moisturizing compounds, vitamins, antioxidants, film-forming polymers, and other desirable ingredients.

The composition of the present invention may contain moisturizing compounds including, but not limited to: N-2-hydroxyethyl urea, additional polyhydric alcohols (also known as polyols) other than 1,2-alkanediols having from 5 to 7 carbon atoms, polyol ethers and esters, low molecular weight polyethylene glycols, lactates, sugars, methyl glucose ethers, sodium pyrrolidone carboxylic acid, sodium hyaluronate, panthenol, and hyaluronic acid, or combinations of the suitable moisturizing compounds.

Examples of the suitable polyols are glycerin (also known as glycerol), propylene glycol (also known as 1,2-propanediol), 1,3-propanediol, 1,2-butanediol, 1,3-butanediol, 2,3-butanediol, 1,5-pentanediol, 2-methyl-2,4,-pentanediol (also known as hexylene glycol), 1,6-hexanediol, diethylene glycol, diglycerin, dipropylene glycol, triethylene glycol, 1,2,3-hexanetriol, 1,2,6-hexanetriol, or combinations of the suitable polyols in any given ratio. Preferred polyols are glycerin, propylene glycol, and hexylene glycol. Examples of the suitable low molecular weight polyethylene glycols (PEG) are PEG 200, PEG 300, PEG 400, PEG 600, and PEG 800. Examples of the suitable lactates are ammonium lactate, sodium lactate, and potassium lactate. Examples of the suitable methyl glucose ethers are methyl gluceth-10 and methyl gluceth-20.

Examples of the suitable vitamins are tocopherol (vitamin E) phosphate, vitamin B.sub.2 and derivatives, vitamin B.sub.3 and derivatives, vitamin C and derivatives thereof, and combinations of the suitable vitamins thereof;

Examples of the suitable antioxidants are green tea extract, grape seed extract, flavonoids, carnosine and derivatives thereof, glutathione, cysteine and derivatives thereof, proline, carnitine and derivatives thereof, and combinations of the suitable antioxidants thereof;

The film forming properties of polymers help maintain active ingredients on the site of application, which may help enhance therapeutic efficacy of the composition. Examples of the suitable film forming polymers are hydroxypropyl cellulose, polyvinylpyrrolidone, polyvinylpyrrolidone vinylacetate copolymer, polyvinyl alcohol, and combinations thereof.

The composition of the present invention may also contain one or more other desirable ingredients including, but not limited to: skin penetration enhancers, herbal extracts, chelating agents, preservatives, colorants, fragrances, and so on. Examples of the suitable herbal extracts are garlic extract, grape seed extract, and onion extract. Examples of the suitable skin penetration enhancers are propylene glycol, dimethyl isosorbide, and N-methyl-1-pyrrolidone. Examples of the suitable chelating agents are EDTA (ethylenediaminetetraacetic acid), EGTA [ethylenebis(oxyethylenenitrilo)tetraacetic acid], and pharmaceutically acceptable salts thereof.

The composition of the present invention may be used for topical treatment of dermatological disorders that are responsive, or potentially responsive, to the dicarboxylic acid therapy. In accordance with the method of treatment of the present invention, a composition containing at least one solubilized dicarboxylic acid is topically applied in a safe and effective amount to skin surfaces of human beings or animals in need of such therapy.

The therapeutic method of the present invention may be used to treat any disorder that is responsive, or potentially responsive, to the dicarboxylic acid therapy. Examples of the disorders that are suitable to be treated include inflammatory lesions on the skin, and certain infectious diseases that may be treated topically.

### Example 1

This example is to formulate a topical solution composition containing 10% solubilized azelaic acid in accordance with the present invention.

| Component | Amount |
|---|---|
| Azelaic acid | 10% |
| Niacinamide | 5% |
| Benzoic acid | 0.2% |
| 1,2-Hexanediol | 25% |
| Water | q.s. to 100% |

Azelaic acid, niacinamide, benzoic acid, and 1,2-hexanediol were added to water. The mixture was kept at about 50.degree. C. while stirring until dissolved. The solution was cooled to room temperature. The pH of the solution was adjusted to about 4.9 using triethanolamine.

### Example 2

This example is to formulate a topical gel composition containing 10% solubilized azelaic acid in accordance with the present invention.

| Component | Amount |
|---|---|
| Azelaic acid | 10% |
| Niacinamide | 5% |
| Benzole acid | 0.2% |
| 1,2-Hexanediol | 25% |
| Hydroxyethyl cellulose | 0.75% |
| Water | q.s. to 100% |

Azelaic acid, niacinamide, benzoic acid, and 1,2-hexanediol were added to water. The mixture was kept at about 50.degree. C. while stirring until dissolved. The solution was cooled to room temperature. The pH of the solution was adjusted to about 4.9 using triethanolamine. Hydroxyethyl cellulose (Klucel, Hercules, Inc., Wilmington, Del.) was combined with the solution while stirring until a clear gel was formed.

### Example 3

Skin Penetration and Bioavailability in the Skin.

The percutaneous penetration and bioavailability of azelaic acid were studied using the gel formulation prepared in Example 2 in accordance with present invention in comparison to Finacea™ (Intendis, Pine Brook, N.J.). The purpose of the study was to describe bioavailability of the solubilized azelaic acid in the gel formulation according to the present invention in the skin and the penetration across the skin. In this respect, the composition from Example 2 was applied in the in-vitro model of horizontal FRANZ-type diffusion cells on the intact complete skin of hairless mice (male, 30-40 days old). An amount of 30 to 50 mg of the following compositions was applied to a skin area of about 1.77 cm²:

1). 15% azelaic acid in Finacea™ (A commercial product);
2). 10% solubilized azelaic acid in the formulation prepared in Example 2.

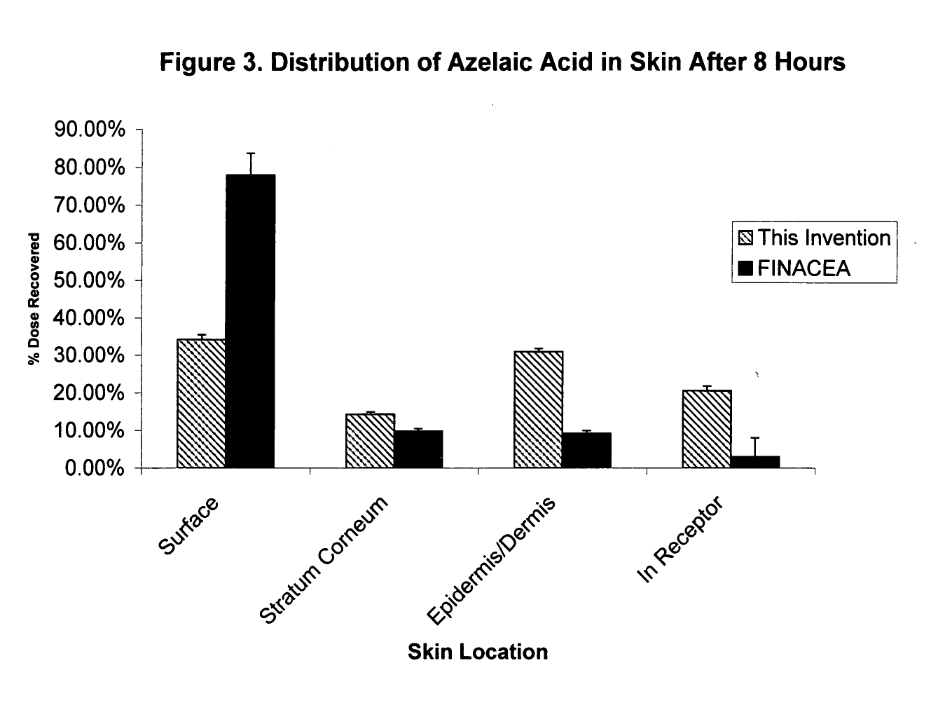

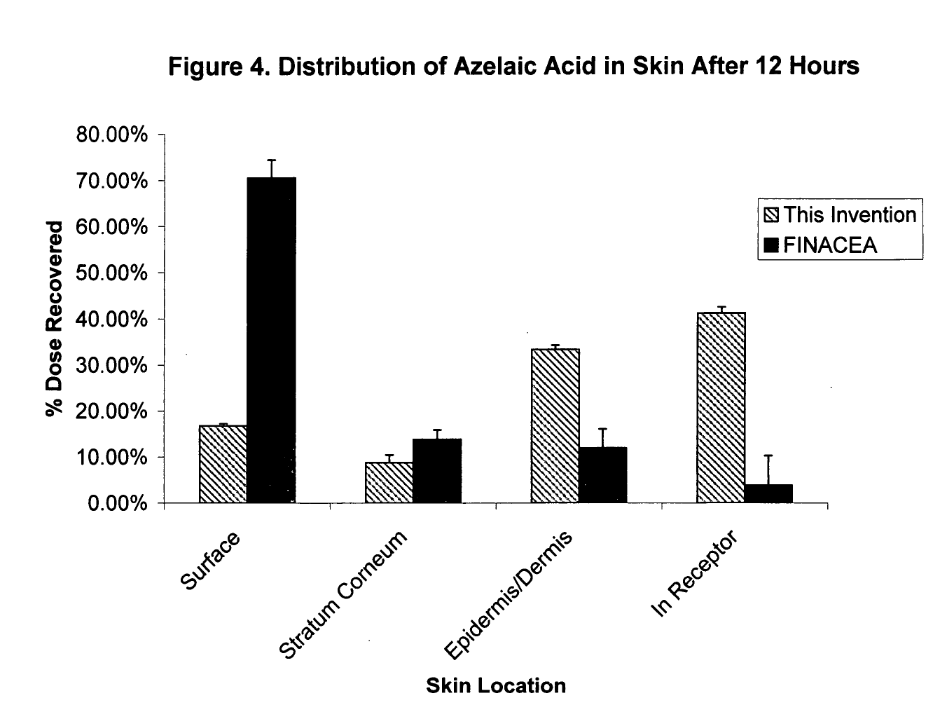

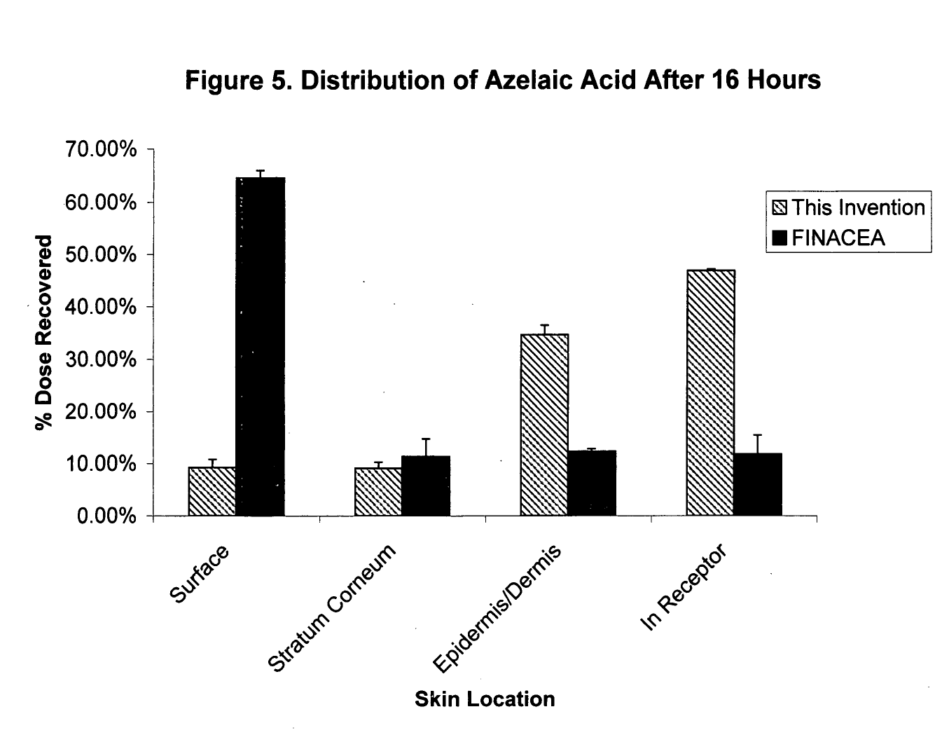

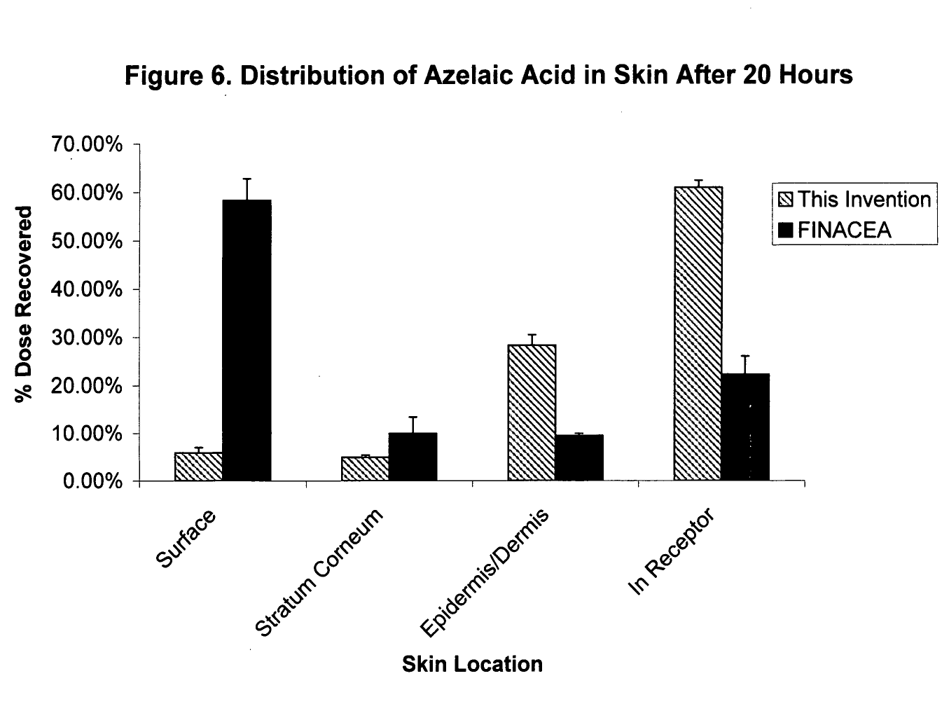

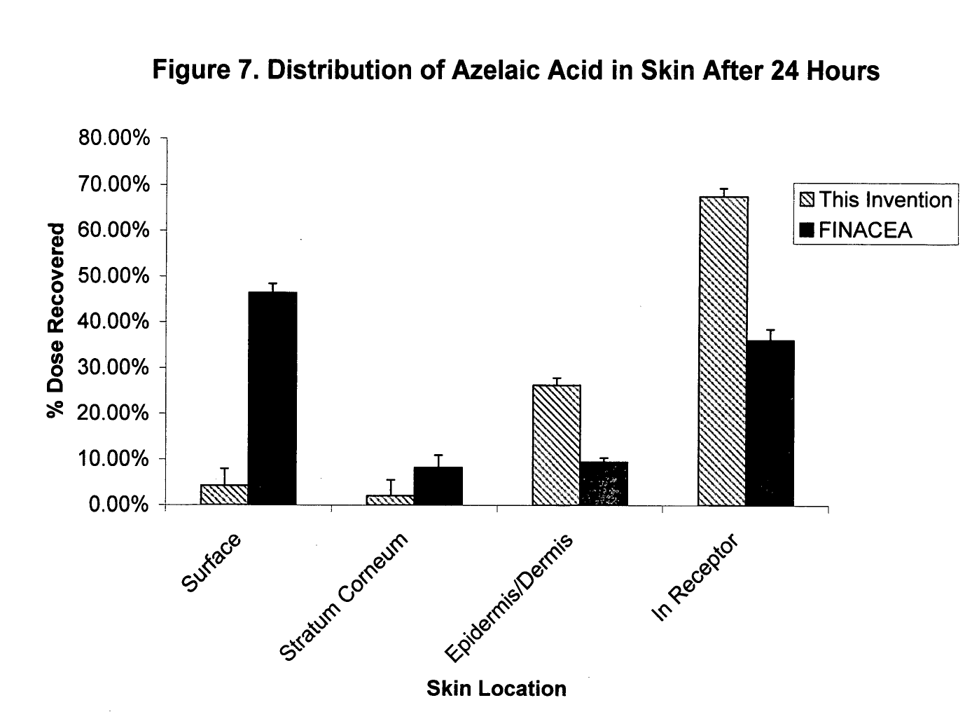

The skin absorption and penetration of the azelaic acid were measured at 8, 12, 16, 20 and 24 hour. At each sampling point, the azelaic acid left on the skin surface (non-penetrated), in the stratum corneum (SC), in the living skin layers and/or cutaneous organs, and in the collection medium (penetrated) were collected and analyzed using HPLC (UV detection at 210 nm, mobile phase: ammonium acetate buffer-methanol 60:40 volume ratio, pH 5.0). The living skin layers and/or cutaneous organs primarily consist of epidermis and dermis layers with subcutaneous fat removed. The experiments were done in triplets. The experimental data are plotted in FIGS. 3 through 7.

The results clearly demonstrate that in comparison to Finacea™, the composition containing solubilized azelaic acid at a concentration of 10% according to the present invention has a significantly higher retention of azelaic acid in the living skin layers and/or cutaneous organs over a period of 24 hours.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

What is claimed is:

1. A topical composition, comprising:
at least one dicarboxylic acid selected from the group consisting of adipic acid, pimelic acid, suberic acid, azelaic acid, sebacic acid, undecanedioic acid, dodecanedioic acid, and combinations thereof;
at lease one 1,2-alkanediol selected from the group consisting of 1,2-pentanediol, 1,2-hexanediol, 1,2-heptanediol, and combination thereof;
a pharmaceutically acceptable carrier;
wherein said dicarboxylic acid and said 1,2-alkanediol are substantially solubilized in said composition.

2. The composition in claim 1 wherein the dicarboxylic acid comprises azelaic acid.

3. The composition in claim 1 wherein the 1,2-alkanediol comprises 1,2-hexanediol.

4. The composition in claim 1 wherein the dicarboxylic acid is azelaic acid and the 1,2-alkanediol is 1,2-hexanediol.

5. The composition of claim 1 wherein the dicarboxylic acid is in an amount within the range of about 1% and about 35% by weight.

6. The composition of claim 1 wherein the dicarboxylic acid is in an amount within the range of about 2% and about 30% by weight.

7. The composition of claim 1 wherein the dicarboxylic acid is in an amount within the range of about 3% and about 25% by weight.

8. The composition of claim 1 wherein the 1,2-alkanediol is in an amount within the range of about 1% and about 50% by weight.

9. The composition of claim 1 wherein the 1,2-alkanediol is in an amount within the range of about 2% and about 40% by weight.

10. The composition of claim 1 wherein the 1,2-alkanediol is in an amount within the range of about 3% and about 35% by weight.

11. The composition of claim 2 wherein the azelaic acid is in an amount within the range of about 1% and about 35% by weight.

12. The composition of claim 2 wherein the azelaic acid is in an amount within the range of about 2% and about 30% by weight.

13. The composition of claim 3 wherein the 1,2-hexanediol is in an amount within the range of about 1% and about 50% by weight.

14. The composition of claim 3 wherein the 1,2-hexanediol is in an amount within the range of about 2% and about 40% by weight.

15. The composition in claim 1 which in the form of a solution, gel, spray, or foam.

16. The composition in claim 1 wherein pH of said composition is within the range of 3.0 and 6.5.

17. A method for treating skin disorders associated with skin inflammation comprising administering a therapeutically safe and effective amount of the composition as claimed in claim 1 to a patient in need of such treatment.

18. The method of claim 17 wherein the skin disorder is acne.

19. The method of claim 17 wherein the skin disorder is rosacea.

20. The method of claim 17 wherein the skin disorder is facial dermatitis.
